(19) United States
(12) Patent Application Publication
(10) Pub. No.: US 2025/0205209 A1
(43) Pub. Date: Jun. 26, 2025

(54) Methods of Treating a Subject for Myalgic Encephalomyelitis/Chronic Fatigue Syndrome (ME/CFS) and Compositions for Use in The Same

(71) Applicant: INCELLDX, INC., Hayward, CA (US)

(72) Inventor: Bruce K. Patterson, Menlo Park, CA (US)

(21) Appl. No.: 18/852,156

(22) PCT Filed: Mar. 29, 2023
(86) PCT No.: PCT/US2023/016692

§ 371 Date: Sep. 27, 2024

Related U.S. Application Data
(60) Provisional application No. 63/325,860, filed on Mar. 31, 2022.

(51) Int. Cl. A61K 31/439; A61K 45/06

(52) U.S. Cl. CPC A61K 31/439; A61K 45/06

(57) ABSTRACT

Methods of treating a subject for Myalgic encephalomyelitis/chronic fatigue syndrome (ME/CFS) are provided. Aspects of the methods include administering to the subject a tropane CCR5/CCL5 interaction inhibitor, either alone or in combination with a statin, to treat the subject for ME/CFS. Also provided are compositions for use in practicing the methods.

### CROSS-REFERENCE TO RELATED APPLICATIONS

Pursuant to 35 U.S.C. § 119 (e), this application claims priority to the filing date of U.S. Provisional Patent Application Ser. No. 63/325,860 filed Mar. 31, 2022, the disclosure of which application is herein incorporated by reference.

### INTRODUCTION

Myalgic Encephalomyelitis/Chronic Fatigue Syndrome (ME/CFS) is a chronic illness that affects anywhere from 836,000 to as many as 2.5 million Americans.¹ ME/CFS especially impacts individuals 40-60 years old, and reports a higher number of affected females than males. However, the illness is not limited to these criteria and any individual may be diagnosed with ME/CFS. The “core” or primary symptoms of ME/CFS include reduced activity level for at least six months, difficulty sleeping or persistent fatigue, and increased ME/CFS symptoms following activity, referred to as post-exertional malaise (PEM). Individuals with ME/CFS also experience cognitive or memory issues, i.e., “brain fog,” and/or orthostatic intolerance, which refers to heightened symptoms when in an upright position. A multitude of additional debilitating symptoms are possible, including pain, tender lymph nodes, recurring sore throat, digestive issues, shortness of breath, and irregular heartbeat. Despite the prominence of the illness and its concerning symptoms, there is no known cause of or laboratory diagnostic for ME/CFS. ¹ https://www.cdc.gov/me-cfs/about/index.html

The CDC has not currently approved any treatments for myalgic encephalomyelitis/chronic fatigue syndrome. As such, present treatments involve controlling patient symptoms. For example, “activity management” or “pacing” is suggested to reduce the frequency of post-exertional malaise. Pacing involves patients developing a better understanding of their ability to be active or exert themselves, also called an “energy envelope,” before they start experiencing symptoms of ME/CFS. In addition, other possible treatments for the symptoms of ME/CFS involve medications such sleep aids, pain-relievers, antidepressants, and anti-anxiety medications, all of which attempt to lessen the painful, chronic symptoms experienced by patients.

CCR5, or chemokine receptor 5, is a G protein coupled receptor involved in the proinflammatory response of the immune system. The CCR5 protein is found on immune cells such as macrophages, activated T cells, and endothelial cells, as well as cells outside of the immune system, such as neurons. CCR5 is recognized for its connection to HIV-1 infection as a co-receptor for the virus. CCL5, chemokine ligand 5, also referred to as RANTES, Regulated upon Activation, Normal T cell Expressed and Secreted, is a protein ligand of CCR5 which may bind to the receptor upon its release. The CCR5-CCL5 pathway is identified to be an important component of mechanisms related to cancer and atherosclerosis, in addition to its involvement in HIV-1 infection.

Maraviroc, also known by brand name Selzentry, is a drug notably used in HIV treatment which functions as a CCR5 antagonist by inhibiting CCR5 receptors on cells. The use of Maraviroc in individuals with R5-tropic HIV-1, a strain of HIV which attacks cells via the CCR5 receptor, is accepted by the U.S. Food and Drug Administration, the European Commission, and Health Canada, among others. Notable clinical trials involving Maraviroc include MOTIVATE 1 and MOTIVATE 2, both of which exhibited Maraviroc's ability to limit the effects of the HIV virus, eliciting lower levels of HIV viral loads and even succeeding in some individuals with R5-tropic HIV-1 who had less favorable starting conditions, such as larger viral loads and reduced CD4 count. Furthermore, a >5 year follow-up of maraviroc use in HIV patients from the MOTIVATE trials presented limited instances of death and other health threats such as hepatic failure and myocardial infarctions, reflecting positively on the safety of maraviroc used in a relatively longer-term.

Myalgic encephalomyelitis/chronic fatigue syndrome is an illness which may affect millions of Americans. ME/CFS can have severe impacts on an individual's life, including increased difficulties socializing and obtaining an education or employment. Thus, there is a great need for an improved treatment method for individuals with myalgic encephalomyelitis/chronic fatigue syndrome.

### SUMMARY

Methods of treating a subject for Myalgic encephalomyelitis/chronic fatigue syndrome (ME/CFS) are provided. Aspects of the methods include administering to the subject a tropane CCR5/CCL5 interaction inhibitor, either alone or in combination with a statin, to treat the subject for ME/CFS. Also provided are compositions for use in practicing the methods.

### DETAILED DESCRIPTION

Methods of treating a subject for Myalgic encephalomyelitis/chronic fatigue syndrome (ME/CFS) are provided. Aspects of the methods include administering to the subject a tropane CCR5/CCL5 interaction inhibitor, either alone or in combination with a statin, to treat the subject for ME/CFS. Also provided are compositions for use in practicing the methods.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

In further describing various aspects of the invention, methods according to embodiments of the invention are discussed first in greater detail, followed by a review of compositions that find use in various embodiments of the methods.

### Methods

As summarized above, methods of treating a subject for Myalgic encephalomyelitis/chronic fatigue syndrome (ME/CFS) are provided. ME/CFS is generally defined as a disorder of uncertain cause that is characterized by persistent, profound fatigue, usually accompanied by impairment and short-term memory or concentration, sore throat, tender lymph nodes, muscle or joint pain, and headache unrelated to any preexisting medical condition, that typically has an onset at between the ages of 30-50 of age. Medline Plus Medical Dictionary. A subject may be diagnosed with CFS using any convenient protocol, such as but not limited to, those described in U.S. Pat. No. 10,215,755, the disclosure of which is herein incorporated by reference. In some instances, the diagnostic protocol is a cytokine hub classification protocol, where in some instances the cytokine hub classification protocol is as described in U.S. Provisional Patent Application Ser. No. 63/325,855 filed Mar. 31, 2022 (the disclosure of which is herein incorporated by reference) and also described in Patterson et al., “Cytokine Hub Classification of PASC, ME-CFS and other PASC-like Conditions,” https://www.researchsquare.com/article/rs-1598634/v1).

Aspects of the methods include administering to the subject a tropane CCR5/CCL5 interaction inhibitor, either alone or in combination with a statin, to treat the subject for ME/CFS.

The tropane CCR5/CCL5 interaction inhibitor employed in methods of the invention may vary. In some instances, the tropane CCR5/CCL5 interaction inhibitor is a compound of formula (I),

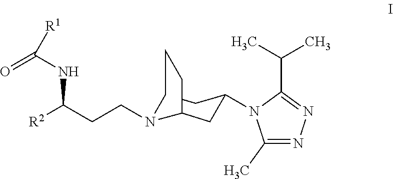

wherein R¹ is C₃₋₆ cycloalkyl optionally substituted by one or more fluorine atoms, or C₁₋₆ alkyl optionally substituted by one or more fluorine atoms, or C₃₋₆ cycloalkylmethyl optionally ring-substituted by one or more fluorine atoms; and
R² is phenyl optionally substituted by one or more fluorine atoms;
or a pharmaceutically acceptable salt or solvate thereof. In some instances, the tropane CCR5/CCL5 interaction inhibitor is a compound of formula (IA),

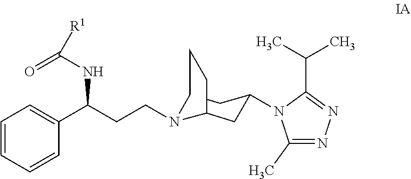

wherein R¹ represents either C₃₋₆ cycloalkyl optionally substituted by one or more fluorine atoms, or C₁₋₆alkyl optionally substituted by one or more fluorine atoms, or a pharmaceutically acceptable salt or solvate thereof.
“C₁₋₆ alkyl” in the definition of R¹ includes straight-chain and branched groups. Examples of alkyl include methyl, ethyl, n-propyl, i-propyl, n-butyl, i-butyl, sec-butyl and t-butyl. “C₃₋₆ cycloalkyl” means cyclopropyl, cyclobutyl, cyclopentyl and cyclohexyl.

The compounds of formula (I) contain a basic center and suitable acid addition salts are formed from acids which form non-toxic salts. Examples include the hydrochloride, hydrobromide, hydroiodide, sulphate, bisulphate, nitrate, phosphate, hydrogen phosphate, acetate, maleate, fumarate, lactate, tartrate, citrate, gluconate, camsylate, succinate, saccharate, benzoate, methanesulphonate, ethanesulphonate, benzenesulphonate, p-toluenesulphonate and pamoate salts. Also of interest in certain embodiments are pharmaceutically acceptable solvates of the compounds of the formula (I) or salts thereof including the hydrates thereof. Also of interest in certain embodiments are polymorphs of the above compounds.

A compound of the formula (I) contains one or more asymmetric carbon atoms and therefore exists in two or more stereoisomeric forms. Of interest in certain embodiments are the individual stereoisomers of the compounds of the formula (I) and, where appropriate, the individual tautomeric forms thereof, together with mixtures thereof. Separation of diastereoisomers may be achieved by conventional techniques, e.g., by fractional crystallization, chromatography or H.P.L.C. of a stereoisomeric mixture of a compound of the formula (I) or a suitable salt or derivative thereof. An individual enantiomer of a compound of the formula (I) may also be prepared from a corresponding optically pure intermediate or by resolution, such as by H.P.L.C. of the corresponding racemate using a suitable chiral support or by fractional crystallization of the diastereoisomeric salts formed by; reaction of the corresponding racemate with a suitable optically active acid or base, as appropriate.

In some instances, R¹ is either C₄₋₆ cycloalkyl optionally substituted by one or two fluorine atoms, or C₁₋₄ alkyl optionally substituted by from one to three fluorine atoms. In some instances, R¹ is either cyclobutyl, cyclopentyl, 4,4-difluorocyclohexyl or 3,3,3-trifluoropropyl. In some instances, R² is phenyl optionally substituted by 1 or 2 fluorine atom(s). In some instances, R² is phenyl or monofluorophenyl. In some instances, R² is phenyl or 3-fluorophenyl.

In some instances, tropane CCR5/CCL5 interaction inhibitor is: N-{(1S)-3-[3-(3-Isopropyl-5-methyl-4H-1,2,4-triazol-4-yl)-exo-8-azabicyclo[3.2.1]oct-8-yl]-1-phenylpropyl)cyclobutanecarboxamide; N-{(1S)-3-[3-(3-Isopropyl-5-methyl-4H-1,2,4-triazol-4-yl)-exo-8-azabicyclo[3.2.1]oct-8-yl]-1-phenylpropyl}cyclopentanecarboxamide; N-{(1S)-3-[3-(3-Isopropyl-5-methyl-4H-1,2,4-triazol-4-yl)-exo-8-azabicyclo[3.2.1]oct-8-yl]-1-phenylpropyl}4,4,4-trifluorobutanamide; N-{(1S)-3-[3-(3-Isopropyl-5-methyl-4H-1,2,4-triazol-4-yl)-exo-8-azabicyclo[3.2.1]oct-8-yl]-1-phenylpropyl}-4,4-difluorocyclohexanecarboxamide; N-{(1S)-3-[3-(3-Isopropyl-5-methyl-4H-1,2,4-triazol-4-yl)-exo-8-azabicyclo[3.2.1]oct-8-yl]-1-(3-fluorophenyl)propyl)-4,4-difluorocyclohexanecarboxamide: or a pharmaceutically acceptable salt or solvate of any thereof; or 4,4-difluoro-N-[(1S)-3-[(1S,5R)-3-(3-methyl-5-propan-2-yl-1,2,4-triazol-4-yl)-8-azabicyclo[3.2.1]octan-8-yl]-1-phenylpropyl]cyclohexane-1-carboxamide (Maraviroc); or a pharmaceutically acceptable salt or solvate of any thereof.

Additional details regarding tropane CCR5/CCL5 interaction inhibitors that may be employed in embodiments of the invention may be found in U.S. Pat. No. 6,667,314 the disclosure of which is herein incorporated by reference.

As reviewed above, the tropane CCR5/CCL5 interaction inhibitor may be administered alone or in combination with a statin. As such, in some instances, the tropane CCR5/CCL5 is administered to the subject as the sole active agent, such that it is not administered in conjunction with other active agents to treat the ME/CFS. In other words, the tropane CCR5/CCL5 interaction inhibitor is not co-administered with another active agent that treats ME/CFS. In other instances, the tropane CCR5/CCL5 is administered in combination with a statin. By “in combination with”, is meant that an amount of the tropane CCR5/CCL5 interaction inhibitor is administered anywhere from simultaneously to up to 5 hours or more, e.g., 10 hours, 15 hours, 20 hours or more, prior to, or after, the statin. In certain embodiments, the tropane CCR5/CCL5 interaction inhibitor and statin are administered sequentially, e.g., where the tropane CCR5/CCL5 interaction inhibitor is administered before or after the statin. In yet other embodiments, the tropane CCR5/CCL5 interaction inhibitor and statin are administered simultaneously, e.g., where the tropane CCR5/CCL5 interaction inhibitor and statin are administered at the same time as two separate formulations, or are combined into a single composition, that is administered to the subject. Regardless of whether the tropane CCR5/CCL5 interaction inhibitor and statin are administered sequentially or simultaneously, as illustrated above, or any effective variation thereof, the agents are considered to be administered together or in combination for purposes of the present invention. Routes of administration of the two agents may vary, where representative routes of administration are described in greater detail below.

Any convenient statin may be employed in embodiments of the invention. Statins are compounds that inhibit HMG-CoA reductase from catalyzing the conversion of HMG-CoA to mevalonate, a rate-limiting step in the cholesterol biosynthetic pathway. Examples of statins that may be used in conjunction with the compositions and methods disclosed herein include, but are not limited to, atorvastatin or atorvastatin calcium (marketed as Lipitor® or Torvast®; see, e.g., U.S. Pat. No. 4,681,893 or 5,273,995) and atorvastatin combinations (e.g., atorvastatin plus amlodipine (marketed as Norvasc®), combination marketed as Caduet®, see, e.g., U.S. Pat. No. 6,455,574; atorvastatin plus CP-529414 (marketed as Torcetrapib®); atorvastatin plus APA-01; atorvastatin plus ezetimibe), cerivastatin (marketed as Lipobay® or Baycol®), fluvastatin (marketed as Lescol®; U.S. Pat. No. 4,739,073), lovastatin (marketed as Mevacor® or Altocor®; see, e.g., U.S. Pat. No. 4,231,938), lovastatin combinations (e.g., lovastatin plus Niaspan®, combination marketed as Advicor®), mevastatin, pitavastatin (marketed as Livalo® or Pitava®), pravastatin (marketed as Pravachol®, Mevalotin®, Selektine®, or Lipostat®; see, e.g., U.S. Pat. No. 4,346,227), pravastatin combinations (e.g., pravastatin plus fenofibrate), rosuvastatin (marketed as Crestor®), rosuvastatin combinations (e.g., rosuvastatin plus TriCor®), simvastatin (marketed as Zocor® or Lipex®; see, e.g., U.S. Pat. Nos. 4,444,784; 4,916,239; and 4,820,850), and simvastatin combinations (e.g., simvastatin plus ezetimibe, combination marketed as Vytorin®, see, e.g., U.S. Pat. No. 7,229,982; simvastatin plus Niaspan®, combination marketed as Simcor®; simvastatin plus MK-0524A, combination referred to as MK-0524B), as well as various pharmaceutically acceptable salts, solvates, salts, stereoisomers, prodrugs derivatives, or nitroderivatives of the compounds listed above. In some cases, such as for example with simvastatin, the active form of the statin is a metabolite formed in the body of a subject following administration. In other cases, statins are administered in their active form.

Where desired, active agents may be administered to a subject as a pharmaceutical composition. The active agent(s) may be administered to the subject using any convenient administration protocol capable of resulting in the desired activity. Thus, the agent can be incorporated into a variety of formulations, e.g., pharmaceutically acceptable vehicles, for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments (e.g., skin creams), solutions, suppositories, injections, inhalants and aerosols. As such, administration of the agents can be achieved in various ways, including oral, buccal, rectal, parenteral, intraperitoneal, intradermal, transdermal, intracheal, etc., administration.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agents can be utilized in aerosol formulation to be administered via inhalation. The compounds of the present invention can be formulated into pressurized acceptable propellants such as dichlorodifluoromethane, propane, nitrogen and the like.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Those of skill in the art will readily appreciate that dose levels can vary as a function of the specific compound, the nature of the delivery vehicle, and the like. Preferred dosages for a given compound are readily determinable by those of skill in the art by a variety of means. With respect to the tropane CCR5/CCL5 interaction inhibitor, any convenient dosage may be employed. For oral or parenteral administration to human patients the daily dosage levels of compounds of formula (I), and their pharmaceutically acceptable salts, may be from 0.01 to 30 mg/kg (in single or divided doses) and in some instances from 0.01 to 15 mg/kg. Thus, tablets may contain 1 mg to 0.5 g of compound for administration singly or two or more at a time, as appropriate. With respect to the statins, any convenient dosage may be employed. In some embodiments, the statin therapy is a low, medium (i.e., moderate), or high intensity statin therapy. In some embodiments, the low intensity statin therapy includes about 5 mg to about 10 mg of simvastatin. In some embodiments, the medium intensity statin therapy includes about 5 mg to about 10 mg of rosuvastatin, about 10 mg to about 20 mg of atorvastatin, about 20 mg to about 40 mg of simvastatin, or about 10 mg to about 20 mg of simvastatin plus about 5 mg to about 10 mg of ezetimibe. In some embodiments, the high intensity statin therapy includes about 20 mg to about 40 mg rosuvastatin, about 40 mg to about 80 mg of atorvastatin, about 80 mg of simvastatin, or about 40 mg to about 80 mg of simvastatin plus about 5 mg to about 10 mg of ezetimibe. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

In practicing embodiments of the invention, active agent compositions may be administered according to any desired dosage, such as once per day, a few or several times per day, or even multiple times per day, depending upon, among other things, the indication being treated and the judgment of the prescribing physician. For example, in some instances, compositions that include one or more active agents may be administered once per day, a few or several times per day, or even multiple times per day, depending upon, among other things, the indication being treated and the judgment of the prescribing physician.

Depending on whether systemic and/or local treatment is chosen, methods of administration may be chosen depending also on the condition being treated and the pharmaceutical composition being administered. Administration of an effective amount (in one or multiple doses) of the subject agent(s) can be done in a variety of ways, including, but not limited to, subcutaneously, intravenously, intraperitoneally, intramuscularly, and direct injection to specified organs or tissues, systemic administration, etc. Administration of the pharmaceutical compositions may be through a single route or concurrently by several routes. As such, the active agent can be administered to a subject via a suitable route of administration and include oral, mucosal (e.g., nasal, sublingual, vaginal, buccal, or rectal), parenteral (e.g., subcutaneous, intravenous, bolus injection, intramuscular, or intra-arterial, intraperitoneal), or transdermal.

In those embodiments where an effective amount of an active agent is administered to the subject, the amount or dosage is effective when administered for a suitable period of time so as to evidence a reduction in one or more symptoms of the target disease. In some instances, an effective amount or dose of active agent will not only slow or halt the progression of the disease condition but will also induce the reversal of the condition, i.e., will cause an improvement in the subject's condition. Where desired, effectiveness of treatment may be assessed using any convenient protocol. Biochemically, by an “effective amount” or “effective dose” of active agent is meant an amount of active agent that will inhibit, antagonize, decrease, reduce, or suppress by about 20% or more, e.g., by 30% or more, by 40% or more, or by 50% or more, in some instances by 60% or more, by 70% or more, by 80% or more, or by 90% or more, in some cases by about 100%, i.e., to negligible amounts, and in some instances reverse, one or more target symptoms of the disease condition.

In some embodiments, the methods include assessing treatment efficacy of the combination therapy. Assessment of treatment efficacy may include evaluation of one or more symptoms of the subject. In some instances, the methods include assessing treatment efficacy by determining whether the subject maintains the ME/CFS pathological type. For example, during treatment of a subject having a ME/CFS pathological type, embodiments of the methods may include further assessing the subject to determine efficacy of the treatment. For example, a subject may be assessed one or more times following treatment to determine whether the subject should still be assigned as having the ME/CFS pathological type, or whether subject no longer has the ME/CFS pathological type. The determination of the pathological type or absence thereof may be employed as a measure or evaluation of the therapeutic treatment regimen being administered to the subject. In such embodiments, the frequency of assaying may vary, such as daily, every two days, weekly, every two weeks, etc. Any convenient ME/CFS pathological type determination may be employed, e.g., as described above in connection with diagnosis of a subject for ME/CFS.

The terms “subject,” “individual,” “host,” and “patient,” are used interchangeably herein and refer to any mammalian subject for whom diagnosis, treatment, or therapy is desired, particularly humans. As reviewed above, by “treatment” it is meant that at least an amelioration of one or more symptoms associated with ME/CFS afflicting the subject is achieved, where amelioration is used in a broad sense to refer to at least a reduction in the magnitude of a parameter, e.g., a symptom, associated with the impairment being treated. As such, treatment also includes situations where ME/CFS, or at least symptoms associated therewith, is completely inhibited, e.g., prevented from happening, or stopped, e.g., terminated, such that the adult mammal no longer suffers from the impairment, or at least the symptoms that characterize the impairment.

### Formulations

Also provided are pharmaceutical compositions for use in treating a subject with ME/CFS. Compositions of embodiments of the invention include a tropane CCR5/CCL5 interaction inhibitor and/or the statin employed in the subject methods. Accordingly, the tropane CCR5/CCL5 interaction inhibitor and/or statin in pharmaceutical compositions, e.g., in the form of a pharmaceutically acceptable salt, can be formulated for oral, topical or parenteral administration for use in the subject methods, as described above. In certain embodiments, e.g., where the compounds are administered as separate formulations (such as in those embodiments where they are administered sequentially), separate or distinct pharmaceutical compositions, each containing a different active agent, are provided. In yet other embodiments, a single formulation that includes both of the tropane CCR5/CCL5 interaction inhibitor and the statin (i.e., one composition that includes both active agents) is provided.

By way of illustration, the tropane CCR5/CCL5 interaction inhibitor and/or the statin can be admixed with conventional pharmaceutically acceptable carriers and excipients (i.e., vehicles) and used in the form of aqueous solutions, tablets, capsules, elixirs, suspensions, syrups, wafers, and the like. Such pharmaceutical compositions contain, in certain embodiments, from about 0.1% to about 90% by weight of the active compound, and more generally from about 1% to about 30% by weight of the active compound. The pharmaceutical compositions may contain common carriers and excipients, such as corn starch or gelatin, lactose, dextrose, sucrose, microcrystalline cellulose, kaolin, mannitol, dicalcium phosphate, sodium chloride, and alginic acid. Disintegrators commonly used in the formulations of this invention include croscarmellose, microcrystalline cellulose, corn starch, sodium starch glycolate and alginic acid.

A liquid composition will generally consist of a suspension or solution of the compound or pharmaceutically acceptable salt in a suitable liquid carrier(s), for example, ethanol, glycerine, sorbitol, non-aqueous solvent such as polyethylene glycol, oils or water, with a suspending agent, preservative, surfactant, wetting agent, flavoring or coloring agent. Alternatively, a liquid formulation can be prepared from a reconstitutable powder.

For example, a powder containing active compound, suspending agent, sucrose and a sweetener can be reconstituted with water to form a suspension; and a syrup can be prepared from a powder containing active ingredient, sucrose and a sweetener.

A composition in the form of a tablet can be prepared using any suitable pharmaceutical carrier(s) routinely used for preparing solid compositions. Examples of such carriers include magnesium stearate, starch, lactose, sucrose, microcrystalline cellulose and binders, for example, polyvinylpyrrolidone. The tablet can also be provided with a color film coating, or color included as part of the carrier(s). In addition, active compound can be formulated in a controlled release dosage form as a tablet comprising a hydrophilic or hydrophobic matrix.

A composition in the form of a capsule can be prepared using routine encapsulation procedures, for example, by incorporation of active compound and excipients into a hard gelatin capsule. Alternatively, a semi-solid matrix of active compound and high molecular weight polyethylene glycol can be prepared and filled into a hard gelatin capsule; or a solution of active compound in polyethylene glycol or a suspension in edible oil, for example, liquid paraffin or fractionated coconut oil can be prepared and filled into a soft gelatin capsule.

Tablet binders that can be included are acacia, methylcellulose, sodium carboxymethylcellulose, poly-vinylpyrrolidone (Povidone), hydroxypropyl methylcellulose, sucrose, starch and ethylcellulose. Lubricants that can be used include magnesium stearate or other metallic stearates, stearic acid, silicone fluid, talc, waxes, oils and colloidal silica.

Flavoring agents such as peppermint, oil of wintergreen, cherry flavoring or the like can also be used. Additionally, it may be desirable to add a coloring agent to make the dosage form more attractive in appearance or to help identify the product.

The compounds of the invention and their pharmaceutically acceptable salts that are active when given parenterally can be formulated for intramuscular, intrathecal, or intravenous administration.

A typical composition for intramuscular or intrathecal administration will be of a suspension or solution of active ingredient in an oil, for example, arachis oil or sesame oil. A typical composition for intravenous or intrathecal administration will be a sterile isotonic aqueous solution containing, for example, active ingredient and dextrose or sodium chloride, or a mixture of dextrose and sodium chloride. Other examples are lactated Ringer's injection, lactated Ringers plus dextrose injection, Normosol-M and dextrose, Isolyte E, acylated Ringer's injection, and the like. Optionally, a co-solvent, for example, polyethylene glycol, a chelating agent, for example, ethylenediamine tetracetic acid, and an anti-oxidant, for example, sodium metabisulphite may be included in the formulation. Alternatively, the solution can be freeze dried and then reconstituted with a suitable solvent just prior to administration.

The compounds of the invention and their pharmaceutically acceptable salts which are active on rectal administration can be formulated as suppositories. A typical suppository formulation will generally consist of active ingredient with a binding and/or lubricating agent such as a gelatin or cocoa butter or other low melting vegetable or synthetic wax or fat.

The compounds of this invention and their pharmaceutically acceptable salts which are active on topical administration can be formulated as transdermal compositions or transdermal delivery devices (“patches”). Such compositions include, for example, a backing, active compound reservoir, a control membrane, liner and contact adhesive. Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. No. 5,023,252, herein incorporated by reference in its entirety. Such patches may be constructed for continuous, pulsatile, or on demand delivery of pharmaceutical agents.

In certain embodiments of interest, the tropane CCR5/CCL5 interaction inhibitor and the statin are administered as a single pharmaceutical formulation, that, in addition to including an effective amount of the tropane CCR5/CCL5 interaction inhibitor and the statin, includes other suitable compounds and carriers, and may also be used in combination with other active agents. The present invention, therefore, also includes pharmaceutical compositions comprising pharmaceutically acceptable excipients. The pharmaceutically acceptable excipients include, for example, any suitable vehicles, adjuvants, carriers or diluents, and are readily available to the public. The pharmaceutical compositions of the present invention may further contain other active agents that are well known in the art.

One skilled in the art will appreciate that a variety of suitable methods of administering a formulation of the present invention to a subject or host. e.g., patient, in need thereof, are available, and, although more than one route can be used to administer a particular formulation, a particular route can provide a more immediate and more effective reaction than another route. Pharmaceutically acceptable excipients are also well-known to those who are skilled in the art and are readily available. The choice of excipient will be determined in part by the particular compound, as well as by the particular method used to administer the composition. Accordingly, there are a wide variety of suitable formulations of the pharmaceutical composition of the present invention. The following methods and excipients are merely exemplary and are in no way limiting.

Formulations suitable for oral administration can consist of (a) liquid solutions, such as an effective amount of the compound dissolved in diluents, such as water, saline, or orange juice: (b) capsules, sachets or tablets, each containing a predetermined amount of the active ingredient, as solids or granules; (c) suspensions in an appropriate liquid; and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

The subject formulations of the present invention can be made into aerosol formulations to be administered via inhalation. These aerosol formulations can be placed into pressurized acceptable propellants, such as dichlorodifluoromethane, propane, nitrogen, and the like. They may also be formulated as pharmaceuticals for non-pressured preparations such as for use in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Formulations suitable for topical administration may be presented as creams, gels, pastes, or foams, containing, or in addition to the active ingredient, and other such carriers that are known in the art to be appropriate.

Suppository formulations are also provided by mixing with a variety of bases such as emulsifying bases or water-soluble bases. Formulations suitable for vaginal administration may be presented as pessaries, tampons, creams, gels, pastes, foams.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

Those of skill in the art will readily appreciate that dose levels can vary as a function of the specific compound, the nature of the delivery vehicle, and the like. Suitable dosages for a given compound are readily determinable by those of skill in the art by a variety of means.

The dose administered to an animal, particularly a human, in the context of the present invention should be sufficient to cause a prophylactic or therapeutic response in the animal over a reasonable time frame. One skilled in the art will recognize that dosage will depend on a variety of factors including the strength of the particular compound employed, the condition of the animal, and the body weight of the animal, as well as the severity of the illness and the stage of the disease. The size of the dose will also be determined by the existence, nature, and extent of any adverse side-effects that might accompany the administration of a particular compound. Suitable doses and dosage regimens can be determined by comparisons to anticancer or immunosuppressive agents that are known to cause the desired growth inhibitory or immunosuppressive response.

Optionally, the pharmaceutical composition may contain other pharmaceutically acceptable components, such a buffers, surfactants, antioxidants, viscosity modifying agents, preservatives and the like. Each of these components is well-known in the art. For example, see U.S. Pat. No. 5,985,310, the disclosure of which is herein incorporated by reference.

Other components suitable for use in the formulations of the present invention can be found in Remington's Pharmaceutical Sciences, Mace Publishing Company, Philadelphia, Pa., 17th ed. (1985). In an embodiment, the aqueous solution of cyclodextrin also contains dextrose, e.g., about 5% dextrose.

### Kits

Also provided are kits that find use in embodiments of the invention. The kits may include a tropane CCR5/CCL5 interaction inhibitor and/or a statin, where the tropane CCR5/CCL5 interaction inhibitor and statin, if present, may be present as separate pharmaceutical compositions or present in a single pharmaceutical composition. The kit components may be present in packaging, which packaging may be sterile, as desired.

Also present in the kit may be instructions for using the kit components. The instructions may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD- or CD-ROM, etc. The instructions may take any form, including complete instructions for how to use the device or as a website address with which instructions posted on the world wide web may be accessed.

The following example(s) is/are offered by way of illustration and not by way of limitation.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

### I. Treatment Study

50 Myalgic encephalomyelitis/chronic fatigue syndrome (ME/CFS) patients and 25 controls are enrolled to receive placebo. Subjects are treated for 6 weeks with a combination of maraviroc 300 mg twice a day and a statin (Pravastatin) 10 mg once a day. Response of biomarkers of the immune system and subjective symptom scores are evaluated.

### II. Cytokine Hub Classification of PASC and PASC-Like Conditions

In some instances, the diagnostic protocol is a cytokine hub classification protocol, where in some instances the cytokine hub classification protocol is as described in U.S. Provisional Patent Application Ser. No. 63/325,855 filed Mar. 31, 2022 (the disclosure of which is herein incorporated by reference) and also described in Patterson et al., “Cytokine Hub Classification of PASC, ME-CFS and other PASC-like Conditions,” https://www.researchsquare.com/article/rs-1598634/v1).

Notwithstanding the appended claims, the disclosure is also defined by the following clauses:

1. A method of treating a subject for Myalgic encephalomyelitis/chronic fatigue syndrome (ME/CFS), the method comprising:
administering to the subject a tropane CCR5/CCL5 interaction inhibitor to treat the subject for ME/CFS.
   2. The method according to Clause 1, wherein the tropane CCR5/CCL5 interaction inhibitor is described by the formula:

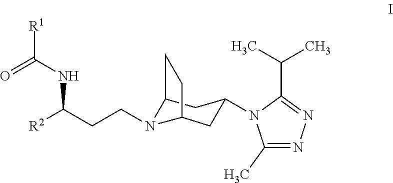

or a pharmaceutically acceptable salt or solvate thereof,
   wherein:
R¹ is C₃₋₆ cycloalkyl optionally substituted by one or more fluorine atoms, or C₁₋₆ alkyl optionally substituted by one or more fluorine atoms, or C₃₋₆ cycloalkylmethyl optionally ring-substituted by one or more fluorine atoms; and
R² is phenyl optionally substituted by one or more fluorine atoms.
   3. The method according to Clause 2, wherein the tropane CCR5/CCL5 interaction inhibitor is described by the formula:

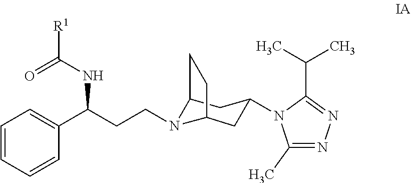

or a pharmaceutically acceptable salt or solvate thereof, wherein:
R¹ is either C₃₋₆ cycloalkyl optionally substituted by one or more fluorine atoms, or C₁₋₆ alkyl optionally substituted by one or more fluorine atoms.
   4. The method according to Clause 3, wherein R¹ is either C₄₋₆ cycloalkyl optionally substituted by one or two fluorine atoms, or C₁₋₄ alkyl optionally substituted by from one to three fluorine atoms.
   5. The method according to Clause 4, wherein R¹ is either cyclobutyl, cyclopentyl, 4,4-difluorocyclohexyl or 3,3,3-trifluoropropyl.
   6. The method according to Clause 2, wherein R² is phenyl optionally substituted by 1 or 2 fluorine atoms.
   7. The method according to Clause 6, wherein R² is phenyl or monofluorophenyl.
   8. The method according to Clause 7, wherein R² is phenyl or 3-fluorophenyl.
   9. The method according to Clause 1, wherein the tropane CCR5/CCL5 interaction inhibitor is selected from the group consisting of:
N-{(1S)-3-[3-(3-Isopropyl-5-methyl-4H-1,2,4-triazol-4-yl)-exo-8-azabicyclo[3.2.1]oct-8-yl]-1-phenylpropyl)cyclobutanecarboxamide;
N-{(1S)-3-[3-(3-Isopropyl-5-methyl-4H-1,2,4-triazol-4-yl)-exo-8-azabicyclo[3.2.1]oct-8-yl]-1-phenylpropyl}cyclopentanecarboxamide;
N-{(1S)-3-[3-(3-Isopropyl-5-methyl-4H-1,2,4-triazol-4-yl)-exo-8-azabicyclo[3.2.1]oct-8-yl]-1-phenylpropyl}4,4,4-trifluorobutanamide;
N-{(1S)-3-[3-(3-Isopropyl-5-methyl-4H-1,2,4-triazol-4-yl)-exo-8-azabicyclo[3.2.1]oct-8-yl]-1-phenylpropyl}-4,4-difluorocyclohexanecarboxamide;
N-{(1S)-3-[3-(3-Isopropyl-5-methyl-4H-1,2,4-triazol-4-yl)-exo-8-azabicyclo[3.2.1]oct-8-yl]-1-(3-fluorophenyl)propyl}-4,4-difluorocyclohexanecarboxamide; and
4,4-difluoro-N-[(1S)-3-[(1S,5R)-3-(3-methyl-5-propan-2-yl-1,2,4-triazol-4-yl)-8-azabicyclo[3.2.1]octan-8-yl]-1-phenylpropyl]cyclohexane-1-carboxamide or a pharmaceutically acceptable salt or solvate of any thereof.
   10. The method according to Clause 9, wherein the tropane CCR5/CCL5 interaction inhibitor is 4,4-difluoro-N-[(1S)-3-[(1S,5R)-3-(3-methyl-5-propan-2-yl-1,2,4-triazol-4-yl)-8-azabicyclo[3.2.1]octan-8-yl]-1-phenylpropyl]cyclohexane-1-carboxamide (maraviroc).
   11. The method according to any of the preceding clauses, wherein the tropane CCR5/CCL5 interaction inhibitor is administered to the subject in combination with a statin.
   12. The method according to Clause 11, wherein the statin is selected from the group consisting of atorvastatin, cerivastatin, fluvastatin, lovastatin, mevastatin, pitavastatin, pravastatin, rosuvastatin, simvastatin, and pharmaceutically acceptable salts, solvates, stereoisomers, or prodrug derivatives thereof.
   13. The method according to any of Clauses 11 or 12, wherein the tropane CCR5/CCL5 interaction inhibitor and statin are administered sequentially to the subject.
   14. The method according to Clause 13, wherein the tropane CCR5/CCL5 interaction inhibitor is administered before the statin to the subject or the tropane CCR5/CCL5 interaction inhibitor is administered after the statin to the subject.
   15. The method according to any of Clauses 11 or 12, wherein the tropane CCR5/CCL5 interaction inhibitor and statin are administered simultaneously to the subject.
   16. The method according to Clause 15, wherein the tropane CCR5/CCL5 interaction inhibitor and statin are administered as separate pharmaceutical formulations to the subject.
   17. The method according to Clause 15, wherein the tropane CCR5/CCL5 interaction inhibitor and statin are administered in the same pharmaceutical formulation to the subject.
   18. The method according to Clause 17, wherein the pharmaceutical formulation comprises an oral formulation.
   19. The method according to Clause 18, wherein the oral formulation comprises a tablet.
   20. The method according to any of the preceding clauses, wherein the subject is a mammal.
   21. The method according to Clause 20, wherein the mammal is a human.
   22. A kit for use in treating a subject for ME/CFS, the kit comprising:
a tropane CCR5/CCL5 interaction inhibitor; and
a statin.
   23. The kit according to Clause 22, wherein the tropane CCR5/CCL5 interaction inhibitor is described by the formula:

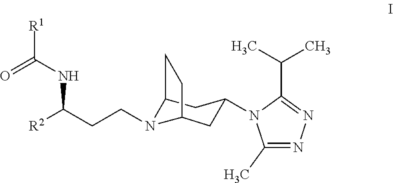

or a pharmaceutically acceptable salt or solvate thereof,
   wherein:
R¹ is C₃₋₆ cycloalkyl optionally substituted by one or more fluorine atoms, or C₁₋₆ alkyl optionally substituted by one or more fluorine atoms, or C₃₋₄ cycloalkylmethyl optionally ring-substituted by one or more fluorine atoms; and
R² is phenyl optionally substituted by one or more fluorine atoms.
   24. The kit according to Clause 23, wherein the tropane CCR5/CCL5 interaction inhibitor is described by the formula:

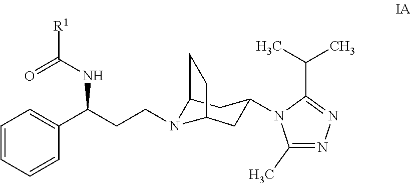

or a pharmaceutically acceptable salt or solvate thereof, wherein:
R¹ is either C₃₋₆ cycloalkyl optionally substituted by one or more fluorine atoms, or C₁₋₆ alkyl optionally substituted by one or more fluorine atoms.
   25. The kit according to Clause 24, wherein R¹ is either C₄₋₆ cycloalkyl optionally substituted by one or two fluorine atoms, or C₁₋₄ alkyl optionally substituted by from one to three fluorine atoms.
   26. The kit according to Clause 25, wherein R¹ is either cyclobutyl, cyclopentyl, 4,4-difluorocyclohexyl or 3,3,3-trifluoropropyl.
   27. The kit according to Clause 23, wherein R² is phenyl optionally substituted by 1 or 2 fluorine atoms.
   28. The kit according to Clause 27, wherein R² is phenyl or monofluorophenyl.
   29. The kit according to Clause 28, wherein R² is phenyl or 3-fluorophenyl.
   30. The kit according to Clause 22, wherein the tropane CCR5/CCL5 interaction inhibitor is selected from the group consisting of:
N-{(1S)-3-[3-(3-Isopropyl-5-methyl-4H-1,2,4-triazol-4-yl)-exo-8-azabicyclo[3.2.1]oct-8-yl]-1-phenylpropyl)cyclobutanecarboxamide;
N-{(1S)-3-[3-(3-Isopropyl-5-methyl-4H-1,2,4-triazol-4-yl)-exo-8-azabicyclo[3.2.1]oct-8-yl]-1-phenylpropyl}cyclopentanecarboxamide;
N-{(1S)-3-[3-(3-Isopropyl-5-methyl-4H-1,2,4-triazol-4-yl)-exo-8-azabicyclo[3.2.1]oct-8-yl]-1-phenylpropyl}-4,4,4-trifluorobutanamide;
N-{(1S)-3-[3-(3-Isopropyl-5-methyl-4H-1,2,4-triazol-4-yl)-exo-8-azabicyclo[3.2.1]oct-8-yl]-1-phenylpropyl}-4,4-difluorocyclohexanecarboxamide;
N-{(1S)-3-[3-(3-Isopropyl-5-methyl-4H-1,2,4-triazol-4-yl)-exo-8-azabicyclo[3.2.1]oct-8-yl]-1-(3-fluorophenyl)propyl}-4,4-difluorocyclohexanecarboxamide; and
4,4-difluoro-N-[(1S)-3-[(1S,5R)-3-(3-methyl-5-propan-2-yl-1,2,4-triazol-4-yl)-8-azabicyclo[3.2.1]octan-8-yl]-1-phenylpropyl]cyclohexane-1-carboxamide
or a pharmaceutically acceptable salt or solvate of any thereof.
   31. The kit according to Clause 30, wherein the tropane CCR5/CCL5 interaction inhibitor is 4,4-difluoro-N-[(1S)-3-[(1S,5R)-3-(3-methyl-5-propan-2-yl-1,2,4-triazol-4-yl)-8-azabicyclo[3.2.1]octan-8-yl]-1-phenylpropyl]cyclohexane-1-carboxamide (maraviroc).
   32. The kit according to any of Clauses 22 to 31, wherein the statin is selected from the group consisting of atorvastatin, cerivastatin, fluvastatin, lovastatin, mevastatin, pitavastatin, pravastatin, rosuvastatin, simvastatin, and pharmaceutically acceptable salts, solvates, stereoisomers, or prodrug derivatives thereof.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”): the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

What is claimed is:

1. A method of treating a subject for Myalgic encephalomyelitis/chronic fatigue syndrome (ME/CFS), the method comprising:
administering to the subject a tropane CCR5/CCL5 interaction inhibitor to treat the subject for ME/CFS.

2. The method according to claim 1, wherein the tropane CCR5/CCL5 interaction inhibitor is described by the formula: 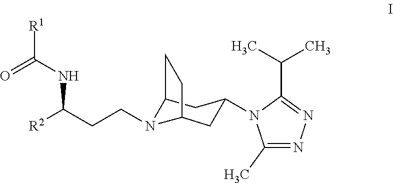
or a pharmaceutically acceptable salt or solvate thereof,
wherein:
R¹ is C₃₋₆ cycloalkyl optionally substituted by one or more fluorine atoms, or C₁₋₆ alkyl optionally substituted by one or more fluorine atoms, or C₃₋₆ cycloalkylmethyl optionally ring-substituted by one or more fluorine atoms; and
R² is phenyl optionally substituted by one or more fluorine atoms.

3. The method according to claim 2, wherein the tropane CCR5/CCL5 interaction inhibitor is described by the formula: 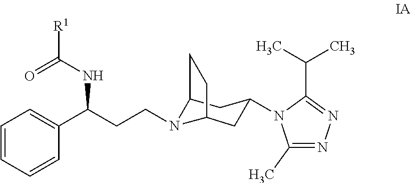
or a pharmaceutically acceptable salt or solvate thereof, wherein:
R¹ is either Ca cycloalkyl optionally substituted by one or more fluorine atoms, or C₁₋₆ alkyl optionally substituted by one or more fluorine atoms.

4. The method according to claim 3, wherein R¹ is either C₄₋₆ cycloalkyl optionally substituted by one or two fluorine atoms, or C₁₋₄ alkyl optionally substituted by from one to three fluorine atoms.

5. The method according to claim 4, wherein R¹ is either cyclobutyl, cyclopentyl, 4,4-difluorocyclohexyl or 3,3,3-trifluoropropyl.

6. The method according to claim 2, wherein R² is phenyl optionally substituted by 1 or 2 fluorine atoms.

7. The method according to claim 6, wherein R² is phenyl or monofluorophenyl.

8. The method according to claim 7, wherein R² is phenyl or 3-fluorophenyl.

9. The method according to claim 1, wherein the tropane CCR5/CCL5 interaction inhibitor is selected from the group consisting of:
N-{(1S)-3-[3-(3-Isopropyl-5-methyl-4H-1,2,4-triazol-4-yl)-exo-8-azabicyclo[3.2.1]oct-8-yl]-1-phenylpropyl)cyclobutanecarboxamide;
N-{(1S)-3-[3-(3-Isopropyl-5-methyl-4H-1,2,4-triazol-4-yl)-exo-8-azabicyclo[3.2.1]oct-8-yl]-1-phenylpropyl}cyclopentanecarboxamide;
N-{(1S)-3-[3-(3-Isopropyl-5-methyl-4H-1,2,4-triazol-4-yl)-exo-8-azabicyclo[3.2.1]oct-8-yl]-1-phenylpropyl}-4,4,4-trifluorobutanamide;
N-{(1S)-3-[3-(3-Isopropyl-5-methyl-4H-1,2,4-triazol-4-yl)-exo-8-azabicycio[3.2.1]oct-8-yl]-1-phenylpropyl}-4,4-difluorocyclohexanecarboxamide;
N-{(1S)-3-[3-(3-Isopropyl-5-methyl-4H-1,2,4-triazol-4-yl)-exo-8-azabicyclo[3.2.1]oct-8-yl]-1-(3-fluorophenyl)propyl}-4,4-difluorocyclohexanecarboxamide; and
4,4-difluoro-N-[(1S)-3-[(1S,5R)-3-(3-methyl-5-propan-2-yl-1,2,4-triazol-4-yl)-8-azabicyclo[3.2.1]octan-8-yl]-1-phenylpropyl]cyclohexane-1-carboxamide or a pharmaceutically acceptable salt or solvate of any thereof.

10. The method according to claim 9, wherein the tropane CCR5/CCL5 interaction inhibitor is 4,4-difluoro-N-[(1S)-3-[(1S,5R)-3-(3-methyl-5-propan-2-yl-1,2,4-triazol-4-yl)-8-azabicyclo[3.2.1]octan-8-yl]-1-phenylpropyl]cyclohexane-1-carboxamide (maraviroc).

11. The method according to any of the preceding claims, wherein the tropane CCR5/CCL5 interaction inhibitor is administered to the subject in combination with a statin.

12. The method according to claim 11, wherein the statin is selected from the group consisting of atorvastatin, cerivastatin, fluvastatin, lovastatin, mevastatin, pitavastatin, pravastatin, rosuvastatin, simvastatin, and pharmaceutically acceptable salts, solvates, stereoisomers, or prodrug derivatives thereof.

13. The method according to any of claims 11 or 12, wherein the tropane CCR5/CCL5 interaction inhibitor and statin are administered sequentially to the subject.

14. The method according to claim 13, wherein the tropane CCR5/CCL5 interaction inhibitor is administered before the statin to the subject or the tropane CCR5/CCL5 interaction inhibitor is administered after the statin to the subject.

15. The method according to any of claims 11 or 12, wherein the tropane CCR5/CCL5 interaction inhibitor and statin are administered simultaneously to the subject.

16. The method according to claim 15, wherein the tropane CCR5/CCL5 interaction inhibitor and statin are administered as separate pharmaceutical formulations to the subject.

17. The method according to claim 15, wherein the tropane CCR5/CCL5 interaction inhibitor and statin are administered in the same pharmaceutical formulation to the subject.

18. The method according to claim 17, wherein the pharmaceutical formulation comprises an oral formulation, preferably a tablet.

19. The method according to any of the preceding claims, wherein the subject is a mammal, preferably a human.

20. A kit for use in treating a subject for ME/CFS, the kit comprising:
a tropane CCR5/CCL5 interaction inhibitor; and
a statin.
